(19) United States
(12) Patent Application Publication
(10) Pub. No.: US 2007/0179356 A1
(43) Pub. Date: Aug. 2, 2007

(54) PROGRAMMABLE DEVICES, SYSTEMS AND METHODS FOR ENCOURAGING THE MONITORING OF MEDICAL PARAMETERS

(75) Inventor: Paul Wessel, Delano, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: GUIDANCE INTERACTIVE HEALTHCARE, INC., Loretto, MN (US)

(21) Appl. No.: 11/617,591

(22) Filed: Dec. 28, 2006

Related U.S. Application Data
(60) Provisional application No. 60/754,399, filed on Dec. 29, 2005.

(51) Int. Cl. A61B 5/00

(52) U.S. Cl. 600/300; 128/905; 128/920

(57) ABSTRACT

Programmable health monitoring devices, systems, and methods for the monitoring of medical parameters are disclosed. An illustrative health monitoring device can include reward circuitry including a reward algorithm or routine for encouraging the monitoring of medical parameters, and a communications interface for transmitting and/or receiving reward data and/or medical data back and forth between the health monitoring device and one or more other devices in communication with the health monitoring device. A display panel can be configured to display various medical and reward parameters, including the user' current and/or past test readings as well as any rewards or incentives earned for complying with the one or more goals determined by the reward algorithm or routine. A user interface can be utilized to program various reward criteria and/or reward codes into the health monitoring device as well as perform other functions.

### CROSS REFERENCE TO RELATED APPLICATION

This application claims the benefit of U.S. Provisional Application No. 60/754,399, filed on Dec. 29, 2005, and entitled “Programmable Incentive Methods Based on Medical Parameters”.

### FIELD

The present invention relates generally to the field of health monitoring. More specifically, the present invention pertains to programmable health monitoring devices, systems, and methods for encouraging the monitoring of medical parameters.

### BACKGROUND

The impact of diabetes-related complications on the population represents a significant portion of healthcare costs worldwide. In the United States alone, more than 18 million individuals suffer from this condition, representing approximately 6 percent of children and adults. Those that suffer from the condition are at a greater risk of cardiovascular related diseases, and typically experience a greater occurrence of amputation and loss of mobility compared to those without the condition. Other physical and psychological factors have also been attributed to diabetes. For example, individuals suffering from diabetes are often more at risk for depression and other behavioral problems. Despite advances in the field, diabetes still remains a significant problem which is expected to rise as the population ages and as more children are diagnosed with an early-onset version of the disease.

Individuals suffering from medical conditions such as diabetes are constantly required to monitor their blood sugar levels to ensure compliance with one or more goals, often under a prescribed medical plan determined by a physician or other healthcare provider. Diabetic patients, for example, are typically required to test their blood glucose levels four or more times per day to ensure that their blood sugar levels are within an acceptable range. In addition to constant monitoring, such individuals are often required to adapt a strict diet and exercise routine as well as undergo insulin therapy in order to maintain their blood sugar levels at acceptable levels.

Blood glucose monitors are frequently employed by individuals suffering from diabetes, hypoglycemia and other blood disorders to determine the amount of glucose contained in the blood stream. These meters typically function by pricking the user's skin with a lancet, and then placing a small capillary blood sample onto a test strip which can then be used by the monitor to sense the amount of glucose within the sample. Once a sample is taken, the monitor then generates a glucose value which can be displayed on a display screen in a desired format (e.g. “mmol/L” or “mg/dL”) based on the user's preference. The readings outputted by the device can then be used by the individual to better manage their condition and, if necessary, take corrective action.

The monitoring of medical parameters such as blood sugar is often a time-consuming and tedious task, requiring the individual to constantly check their condition to ensure that they are in compliance with the goals of their prescribed medical plan. The desire to perform such monitoring is often counterbalanced or outweighed by the pain and inconvenience associated with such tests. For example, for diabetics who are required to test their blood sugar levels multiple times throughout the day, the desire to perform such self-testing may be reduced by the pain associated with pricking their finger with a lancet. In some cases, psychological factors such as the individual's self-esteem or the psychosocial stigma associated with performing self-tests in public may also affect the individual's desire to perform such monitoring. These physical and mental hurdles are particularly acute in children, who frequently experience diabetes burnout at an early age if not sufficiently motivated to continue with their testing regimen.

### BRIEF SUMMARY

The present invention relates generally to programmable health monitoring devices, systems, and methods for encouraging the monitoring of medical parameters. An illustrative health monitoring device can include medical circuitry adapted to sense one or more medical parameters such as blood glucose or blood pressure, and reward circuitry adapted to run a reward algorithm or routine for encouraging the monitoring of one or more medical parameters by the user. The reward circuitry can include a reward criteria database containing one or more programmed goals associated with the user's medical condition. During use, the reward algorithm or routine can be configured to formulate a reward or incentive based on the user's compliance with the one or more goals as determined by the reward criteria. A communications interface can be utilized to transmit and receive reward data and/or medical data back and forth between the health monitoring device and one or more remote devices.

The health monitoring device can be equipped with a display panel including one or more display screens that can be used to display medical test data as well as various reward and motivation messages. In some embodiments, the health monitoring device can further include one or more light sources for providing the user with a visual indication of their compliance with the one or more goals determined by the reward criteria as well as any rewards or incentives that have or will be earned based on their compliance with those goals. Other status indicators such as an audible alarm outputted by a speaker or tactile feedback provided by a vibration element within the health monitoring device may also be provided to the user, if desired.

The health monitoring device can be used as part of a system for encouraging the monitoring of one or more medical parameters. In certain embodiments, for example, the health monitoring device can be connected to at least one remote device via a wired or wireless communications link, allowing various medical and/or reward data to be transmitted back and forth between the health monitoring device and each remote device. In an on-line based system, the health monitoring device can be connected to one or more web-sites and/or other remote devices via an Internet or intranet connection. Examples of remote devices that can be connected to the health monitoring device can include the computer system of a remote entity such as a pharmacy, medical supply store, health clinic, health club facility, or fitness center. The remote device can also include other monitoring devices such as blood pressure monitors, blood oxygen monitors, and/or heart monitors as well as other external devices such as personal computers, laptop computers, hand-held computers, cellular telephones, pagers, television set or cable boxes, video game consoles, and/or digital media players.

An illustrative method of providing individuals with a reward or incentive for monitoring one or more medical parameters with the health monitoring device can include the steps of receiving reward criteria data into the health monitoring device, comparing the reward criteria data against one or more stored medical parameters sensed by the health monitoring device, determining whether one or more goals of the reward criteria data have been satisfied, prompting the user to redeem one or more rewards or incentives based at least in part on their compliance with the one or more goals, and then downloading the reward or incentive into the health monitoring device and/or transmitting the reward or incentive to one or more other devices.

### BRIEF DESCRIPTION OF THE DRAWINGS

FIG. 1 is a perspective view of a health monitoring device in accordance with an illustrative embodiment;

FIG. 2 is a schematic view showing several illustrative components of the health monitoring device of FIG. 1;

FIG. 3 is a schematic view showing the reward circuitry and medical circuitry for the health monitoring device of FIG. 1;

FIG. 4 is a diagrammatic view of an illustrative optical system for monitoring the illumination status of the LED's used by the health monitoring device of FIG. 1;

FIG. 5 is a flow diagram of an illustrative method of providing users with a reward or incentive for monitoring one or more medical parameters using a health monitoring device;

FIG. 6 is a flow diagram of an illustrative method of providing a reward status indicator to a user using a health monitoring device;

FIG. 7 is a flow diagram of an illustrative method of triggering a reward or incentive using the internal clock circuitry of the health monitoring device;

FIGS. 8-10 are several perspective views of the health monitoring device of FIG. 1, showing the steps of providing a visual indicator to the user indicating that a reward or incentive has or will be earned;

FIG. 11 is a diagrammatic view showing an illustrative reward system for rewarding a user for monitoring one or more medical parameters using a health monitoring device;

FIG. 12 is a diagrammatic view showing an illustrative on-line reward system for rewarding a user for monitoring one or more medical parameters using a health monitoring device;

FIG. 13 is a diagrammatic view showing an illustrative implementation of the on-line reward system of FIG. 12; and

FIG. 14 is a diagrammatic view showing an illustrative vehicle reward system for rewarding a driver for monitoring one or more medical parameters using a health monitoring device.

### DETAILED DESCRIPTION

The following description should be read with reference to the drawings, in which like elements in different drawings are numbered in like fashion. The drawings depict several illustrative embodiments, and are not intended to limit the scope of the invention. While the devices, systems, and methods are frequently described herein with respect to continuous blood glucose monitors, it should be understood that other medical and non-medical devices can incorporate one or more of the features described herein. Examples of other devices can include, but are not limited to, blood pressure monitors, blood oxygen monitors, heart monitors, spirometers, insulin pumps, pedometers, scales, shoes, exercise or fitness equipment, personal computers, laptop computers, hand-held computers, cellular telephones, pagers, television set or cable boxes, video game consoles, and digital media players.

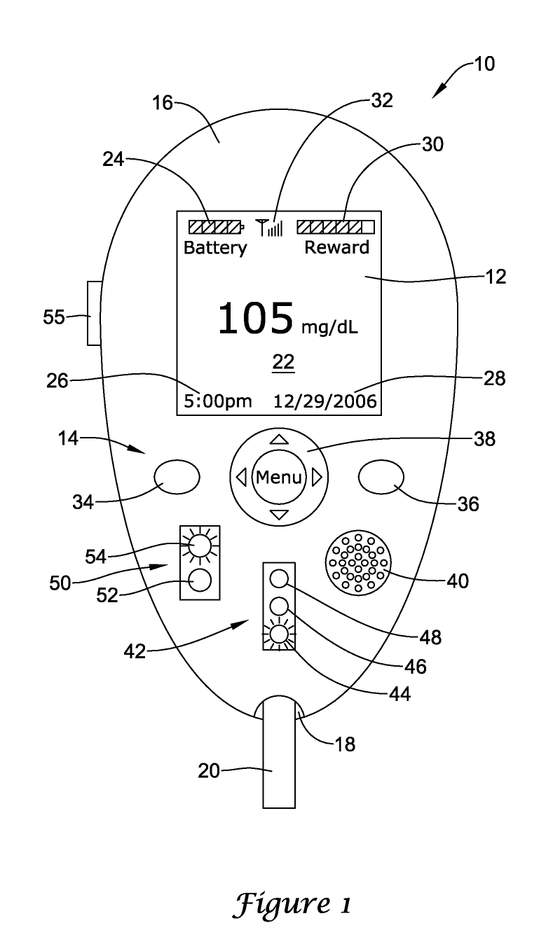

Referring now to FIG. 1, a health monitoring device 10 in accordance with an illustrative embodiment will now be described. Device 10, illustratively a blood glucose monitor for use by a diabetic or hypoglycemic user, can include a display panel 12 for displaying various operational status and monitoring information, and a user interface 14 which can be used to enter, and in some cases program, various commands into the device 10. The display panel 12 and user interface 14 may be inset within a housing 16 of the device 10 having a port 18 adapted to receive a test strip 20 containing a sample of blood obtained from the user. Once inserted into the port 18, the test strip 20 can be used by the device 10 to determine the user's current glucose levels, which can then be outputted as a result (e.g. “105 mg/dL”) on a display screen 22, as shown.

The display panel 12 can include a liquid crystal display (LCD), light emitting diode (LED) panel, touchscreen, or other suitable means for displaying information to the user. In some embodiments, the display panel 12 can be configured by the user interface 14 and/or remotely by an external computing device to display both text and graphics simultaneously on the display screen 22. Alternatively, and in other embodiments, the display panel 12 can be configured, either locally and/or remotely, to display only text characters on the display screen 22. While only a single display panel 12 is depicted in FIG. 1, it should be understood that multiple display panels may be utilized to display information. For example, the monitoring device 10 can include a first display panel for displaying one or more current and/or past glucose test readings obtained from the user, and a second display panel for displaying any accrued rewards or incentives the user has or will earn as a result of complying with the goals of their prescribed medical plan, as discussed further herein.

The display panel 12 can be configured to provide other information to the user, including a progression indicator 24 indicating the current charge-status of the batteries, a time indicator 26 indicating the current time, and a date indicator 28 indicating the current date. A second progression indicator 30 can also be provided on the display screen 22 indicating when a reward or incentive has or will be earned. In those embodiments in which the monitoring device 10 is a wireless device, a signal strength meter 32 may display the signal strength of the wireless connection between the monitoring device 10 and another remote device.

The user interface 14 can include a number of keys, buttons, dials and/or other means for inputting, and in some cases programming, various commands into the monitoring device 10. A first set of buttons 34,36, for example, can be utilized to set the current display mode to cause the display panel 12 to switch back and forth between a number of different display screens. Selection of button 34, for example, can cause the monitoring device 10 to switch between a display screen which shows the user's most recent glucose reading(s), a display screen which shows one or more previously stored glucose readings or an averaged glucose reading, and a display screen that can be used to show any accrued rewards or incentives earned and/or other useful information pertinent to the user's medical condition. Selection of button 36, in turn, can cause the monitoring device 10 to toggle the format in which the device 10 displays glucose level readings. For example, selection of button 36 can cause the monitoring device 10 to toggle between the display of glucose level readings between a “mmol/L” format and a “mg/dL” format.

A circle-wheel button 38 can be further provided to permit the user to access other display screens and/or functionality within the monitoring device 10 or an external computing device, including a setup screen that can be used to set the current date and time, to clear any previously stored glucose level readings sensed by the device 10 and/or to set various interface parameters used by the device 10 to communicate with other devices. The circle-wheel button 38 can also be utilized to access other functionality including a communications screen that can be used to view and/or send messages and other data back and forth between the monitoring device 10 and another remote device.

The monitoring device 10 can be further equipped with a speaker 40 that can be used to prompt the user to perform certain tasks as well as to aurally provide the user with information regarding their current health status. The speaker 40, for example, can be configured to produce an audible alarm tone reminding the user to take a new glucose level reading, informing the user that a new reward or incentive has been earned, and/or prompting the user to perform some other task such as transmitting data to another device. Other audible alarm tones may also be provided warning the user that the battery level is low or alerting the user that a message has been received and is ready for viewing. In some embodiments, the speaker 40 can be used to output voice messages received by the device 10. For example, the speaker 40 can be used to output MP3, WAV, or other such audio files received by the user's caregiver or physician educating the user about their current health status, and to provide motivational messages to encourage the user's compliance with their prescribed medical plan.

The pitch and/or volume of the alarm tones can be varied to notify the user of the importance to take action, or to differentiate between types of alerts. For example, the device 10 can be configured to output an audible alarm tone at a first pitch prompting the user to take a glucose level reading, and another alarm tone at a second, discemable pitch informing the user that a message and/or new reward code is available for viewing. The loudness of the alarm tones may vary depending on the urgency in which action must be taken. For example, the loudness of the alarm tone outputted by the speaker 40 can be increased as the time from the user's last glucose level reading increases, thus providing the user with feedback of the urgency to take a new measurement. The time between the alarm tones can also be increased, informing the user of the urgency to take action. In some embodiments, the monitoring device 10 can be further equipped with an internal vibration element that can be used to provide the user with tactile feedback of the urgency to take measurements.

A number of light sources (e.g. LED's) inset within the housing 16 can be used to provide the user with status information on their compliance with the programmed goals of their prescribed medical plan as well as information on whether any rewards or incentives have or will be earned. A first array 42 of LED's within the housing 16, for example, can provide the user with visual feedback on the user's compliance with their blood glucose monitoring. The array 42 of LED's can be arranged in a format that can be quickly and easily understood by the user. In the illustrative embodiment depicted in FIG. 1, for example, the array 42 of LED's can include three separate LED's 44,46,48 arranged in a stop-light configuration, with the illumination of a green LED 44 used to indicate full compliance, a yellow LED 46 used to indicate that action may be needed, and a red LED 48 used to indicate that prompt action is necessary. The illumination of the green LED 44 may indicate, for example, a blood glucose level of between 80-90 mg/dL whereas the illumination of the yellow and red LED's 46,48 may indicate blood glucose levels of between 90-100 and 101+, respectively. Other light configurations can also be utilized to provide the user with a visual indicator of their compliance. In certain embodiments, the monitoring device 10 can be programmed to assign specific ranges to one or more of the LED's 44,46,48. If, for example, the user desires to assign a glucose range different than the default range pre-programmed within the monitoring device 10, the user may select the appropriate button or buttons on the user interface 14 to adjust the range.

A second array 50 of LED's can be provided to alert the user of any unused rewards or incentives that have or will be earned as a result of compliance with the goals of the user's prescribed medical plan. The second array 50 of LED's can include, for example, a first LED 52 for informing the patient whether they have any rewards which have not been claimed, and a second LED 54 that can be used to inform the user whether a reward or incentive is forthcoming. The first LED 52, for example, can be illuminated when the user has received a new reward code as a result of successfully monitoring their blood glucose levels for a pre-determined period of time. The second LED 54, in turn, can be illuminated when a new reward or incentive is impending based on the user's compliance with various reward criteria programmed within the monitoring device 10.

The appearance and/or blink rate of the LED's 52,54 can be altered to further indicate the status of any rewards or incentives earned. For example, the first LED 52 can be configured to output a first color (e.g. green) indicating that a reward code or incentive has been earned whereas the LED 52 can be configured to output a different color (e.g. red) indicating that a reward or incentive has not been earned. Alternatively, and in other embodiments, the blink rate of the LED 52 may be adjusted depending on the number of unused reward codes or incentives that have been earned. For example, the LED 52 may blink once indicating that one unused reward code or incentive has been earned, twice indicating that two unused reward codes or incentives have been earned, and so forth. The appearance and/or blink rate of the second LED 54 can be similarly adjusted to provide the user with an indication of when an upcoming reward or incentive will be earned. For example, the blink rate of the second LED 54 can be made proportional to the time remaining for the next reward or incentive to be earned.

An external communications port 55 can be utilized to connect the monitoring device 10 to an external computing device such as a personal computer, laptop computer, hand-held computer, cellular telephone video game console, or digital media player. In certain embodiments, for example, the external communications port 55 may be utilized to connect the monitoring device 10 to another monitoring device such as a blood pressure monitor, allowing the user to transmit and/or receive data back and forth between the two monitoring devices. In some cases, the external communications port 55 may permit other devices such as a printer to be connected to the monitoring device 10, if desired.

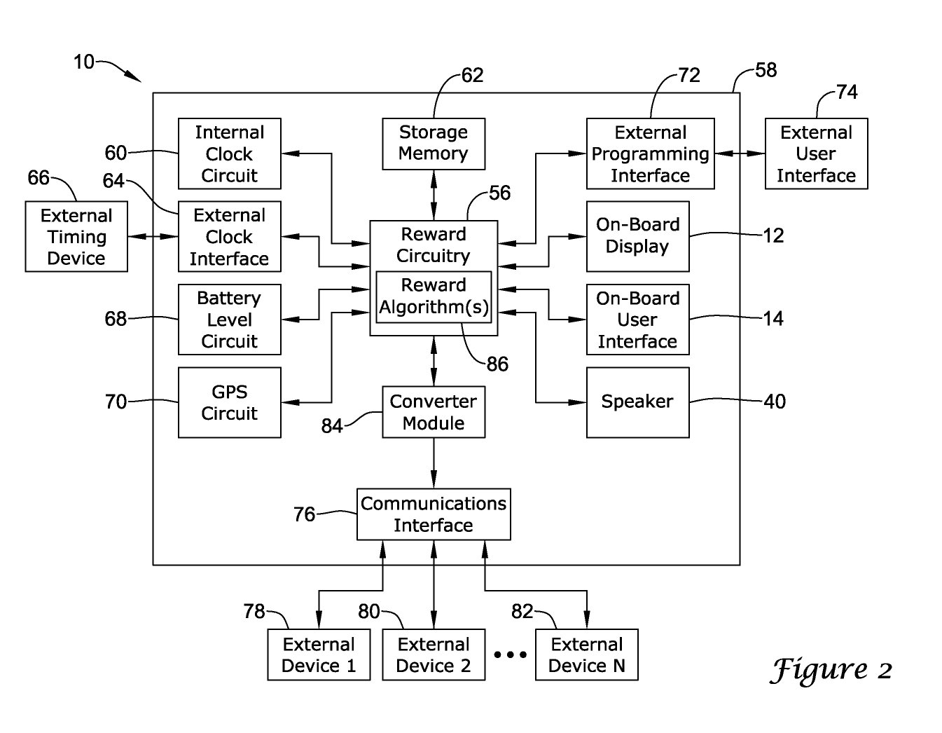

FIG. 2 is a schematic view showing several components of the health monitoring device 10 of FIG. 1. As can be seen in FIG. 2, the monitoring device 10 can include reward circuitry 56 electrically connected to a number of other components located on a circuit board 58 within the device housing 16. An internal clock circuit 60 can used to maintain the current time and date as well as for timing the duration between glucose level readings. The clock circuit 60 can be configured to provide a date and time stamp for each glucose level reading, which can be stored along with the reading within a storage memory 62 such as a RAM, EEPROM, or flash memory. In use, the clock circuit 60 can be used determine the type and/or frequency of rewards or incentives provided to the user. For example, the clock circuit 60 can be used to produce a particular reward on the user's birthday or if the user promptly performed a reading every four hours as required by their prescribed medical plan.

An external clock interface 64 can be used to synch the time and/or date of the monitoring device 10 with another external timing device 66, if desired. For example, in some embodiments the external clock interface 64 can be used to synch the monitoring device 10 with the timer of another monitoring device such as a heart monitor, blood pressure monitor, blood oxygen monitor, spirometer, or insulin pump. In some cases, the external clock interface 64 can be used to obtain the time and date automatically from a remote source such as from a radio signal.

The external clock data received via the external clock interface 64 can be compared against the time and date maintained by the internal clock circuit 60 to determine the user's behavioral compliance patterns for their prescribed medical plan. In some embodiments, for example, the external clock data can be used to determine trends associated with the user's monitoring of their medical parameters. If, for example, the user is habitually late in performing their testing regimen at a particular time of the day such as in the evening, the external clock data can be compared against the date and time stamps associated with their past readings to determine an alternative time to perform such tests.

A battery level circuit 68 can be used to monitor the level of the batteries used to power the monitoring device 10. The battery level circuit 68, for example, can measure the charge of the batteries and output a message or status indicator (e.g. via the progression indicator 24 on the display screen 22) notifying the user that the batteries are low and require replacement or recharging. In some embodiments, the battery level circuit 68 may further output a message or status indicator informing the user that the batteries are being recharged when rechargeable batteries are used.

A GPS circuit 70 can be configured to receive a global positioning signal (GPS) that can be used to track the location of the monitoring device 10. In some embodiments, for example, the GPS circuit 70 can be used to monitor whether the user is out of range from receiving healthcare from their healthcare provider, or is located near a store where medical supplies can be purchased. For example, the signals received by the GPS circuit 70 can be utilized in conjunction with mapping software and/or hardware to provide the user with directions to the closest medical supply store for purchasing needed supplies. In some cases, the GPS circuit 70 can be used to notify the user where to redeem a reward or incentive that has been earned as a result of their compliance with the goals of their prescribed medical plan.

An external programming interface 72 can be used to connect the monitoring device 10 to an external user interface 74 to permit the user to interact with, and in some cases program, the device 10 from a remote location and/or via another device. For example, in some embodiments the external programming interface 72 can be used to program the monitoring device 10 from a keyboard, keypad, or other suitable means for inputting data to the device 10. In some cases, the external programming interface 72 may permit reward codes to be programmed into the device 10 by the manufacturer of the device 10, or by the user's healthcare provider, insurance provider, or caregiver. The external programming interface 72 may permit either wired or wireless transmission of programming data via either a bidirectional or asynchronous port.

The monitoring device 10 can further include a communications interface 76 that can be used to transmit and receive various commands and data back and forth between one or more external devices 78,80,82 in communication with the device 10. Examples of external devices that can be connected to the monitoring device 10 via the communications interface 76 can include, but are not limited to, personal computers, laptop computers, hand-held computers (e.g. PDA, BLUETOOTH, PALM-PILOT), cellular telephones, pagers, television set or cable boxes, video game consoles, digital media players (e.g. IPOD, MP3 or MPEG players), point of sale devices, bar code readers, and vehicle controllers. Other healthcare devices such as blood pressure monitors, blood oxygen monitors, heart monitors, spirometers, insulin pumps, and pedometers can also be connected to the monitoring device 10 via the communications interface 76, if desired. In some cases, the communications interface 76 may permit other devices such as a printer to be connected to the monitoring device 10, allowing test data and reward information to be printed.

Connection between the communications interface 76 and the external devices 78,80,82 can be accomplished via a wired communications link such a USB cable, IEEE394 cable, Ethernet cable, serial (e.g. RS232) cable, parallel cable, or optical cable. Connection to the external devices 78,80,82 can also be accomplished with a wireless communications link such as via an RF signal (e.g. 802.11a, 802.11b, 802.11g, Bluetooth, Zigbee, etc.) or infrared signal (e.g. MRDA). In some embodiments, connection to one or more of the external devices 78,80,82 can be accomplished via the Internet through a dial-up connection, DSL connection, cable broadband connection, or the like. A converter module 84 can be used to convert and, in some cases encrypt, data sent back and forth over the connection lines. For example, the converter module 84 can be used to convert glucose level readings into a different and more secure format that can then be transmitted and deciphered by another device in communication with the monitoring device 10.

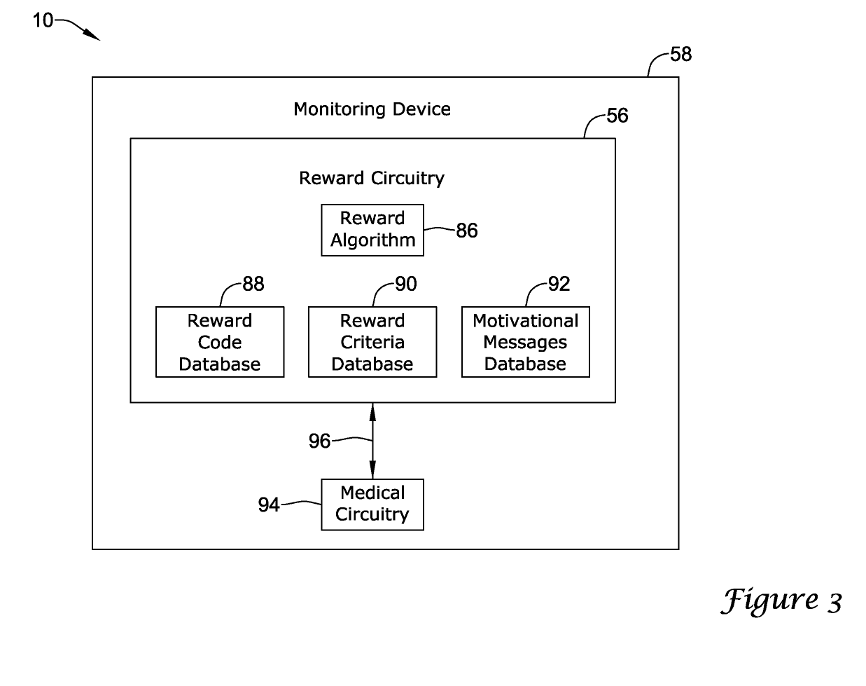

The reward circuitry 56 can be configured to run one or more reward algorithm or routines 86 that can be used to monitor the user's progress in monitoring their glucose levels and formulate rewards or incentives based on the user's compliance with reward criteria programmed within the monitoring device 10. As can be further seen in FIG. 3, the reward circuitry 56 can include a reward code database 88 such as a look-up table containing one or more reward codes that can be provided when the user satisfies one or more reward criteria from a reward criteria database 90. For example, for diabetic users, the reward circuitry 56 can be internally or externally configured to provide the user with one or more reward codes when the user successfully completes a series of glucose level readings on-time, when the user's glucose levels are maintained within a certain range for a period of time, or other such goal. The reward codes contained within the reward code database 88 can be provided as bar codes, alpha numeric characters, hexadecimal characters, or other type of encrypted code. In use, the reward algorithm or routine 86 can be configured to perform various computational functions to determine whether the user's monitoring of one or more medical parameters satisfies the reward criteria programmed within the database 90.

The reward circuitry 56 can further include a motivational messages database 92 containing a number of motivational messages that can be used to encourage patient compliance with the goals of their prescribed medical plan. In certain embodiments, for example, the reward circuitry 56 can be configured to generate motivational messages triggered based on the user's compliance with the reward criteria, which can then be displayed on the display screen as a text message and/or graphic or outputted from the speaker as an audible message. The reward circuitry 56 can be configured to output motivational messages at certain times of the day and/or in response to the user's behavioral patterns. For example, if the reward circuitry 56 determines that the user is one hour past due in taking a glucose level reading, the reward circuitry 56 may display one or more motivational messages on the display screen encouraging the user to take a measurement.

Other types of messages may also be stored within the database 92, which can then be provided based on the user's compliance with the reward criteria, the user's personal information, as well as other factors. For example, and in some embodiments, the database 92 may also contain promotional advertising messages or special offers that can be provided to the user based on criteria programmed within the monitoring device 10 and/or criteria provided by another external computing device in communication with the monitoring device 10. In one illustrative embodiment, advertising messages may be pre-programmed within the monitoring device relating to a particular retailer's products. In exchange for such advertising, the retailer can be assessed a sponsorship fee.

The reward circuitry 56 can be optically isolated from the medical circuitry 94 or can be formed integrally therewith. During operation, the medical circuitry 94 can be configured to transmit medical data to the reward circuitry 56 via a communications link 96, which can then be received and stored within the memory along with a date and time stamp corresponding to the date and time the measurements were taken. The reward circuitry 56 containing the reward codes, reward criteria, and motivational messages can be implemented as either hardware and/or software, and can be programmed via the on-board user interface, the external user interface, an external device such as a computer, and/or via the Internet. In some embodiments, the reward codes, reward criteria, promotional and motivational messages can be pre-programmed at the factory, at a servicing kiosk, or other such location. Programming of the reward codes, reward criteria, and motivational messages can be accomplished via a wireless or wired connection, remotely or locally, by setting a dip switch, or by other means.

In some embodiments, the reward circuitry 56 can be configured to convert reward codes to another format. For example, the reward circuitry 56 can be configured to take reward codes provided as an alphanumeric message and convert that message into another electronic format such as an audible message, a printer file, etc. Conversion of the reward codes can be accomplished, for example, using the converter module 84 described above with respect to FIG. 2, although other means for converting the codes are possible. In some cases, the reward circuitry 56 may permit multiple reward codes to be combined together for redemption, if desired.

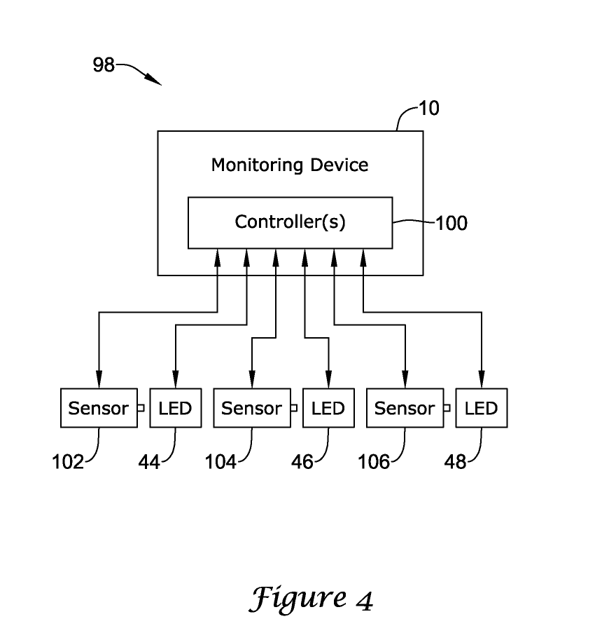

FIG. 4 is a diagrammatic view of an illustrative optical system 98 for self-checking the illumination status of the LED's used by the health monitoring device 10. As shown in FIG. 4, the monitoring device 10 may further include a separate controller 100, or multiple controllers, in communication with each of the LED's 44,46,48. The controller 100, may comprise, for example, a part of the reward circuitry 56 or medical circuitry 94 adapted to send signals to each of the LED's 44,46,48, causing them to illuminate in a desired manner based on computations made by the reward algorithm or routine 86.

To provide a level or redundancy to the system 98 in the event one or more of the LED's 44,46,48 burns out or otherwise becomes inoperable, an optical detector 102,104,106 located adjacent to each LED 44,46,48 can be configured to send a signal back to the controller 100 informing the controller 100 of the illumination status of each of the LED's 44,46,48. For example, the optical detectors 102,104,106 may send signals back to the controller 100 indicating whether the LED's 44,46,48 are currently illuminated when activated. In some embodiments, the optical detectors 102,104,106 can be configured to sense other information such as the wavelength of light outputted by the LED's 44,46,48 to determine whether the LED's 44,46,48 are functioning properly. The optical detectors 102,104,106 can be separate components from the LED's 44,46,48, or can be formed integrally with the LED's 44,46,48. Although optical detectors 102,104,106 can be utilized to sense whether the LED's 44,46,48 are functioning properly, it should be understood that other suitable means for checking the operational status of the LED's 44,46,48 may also be employed, if desired.

The comparison of the illumination status of the LED's 44,46,48 can be accomplished using a single controller or multiple controllers. When multiple controllers are employed, the monitoring information obtained by each controller can be compared against each other to determine whether the LED's are not functioning properly. If the status of the LED's is the same, an acceptable monitoring condition exists. When a single controller is employed, four inputs can be provided to the controller for each LED and corresponding sensor. For example, a first input pin of the controller can receive a first set of inputs from the LED and sensor whereas a second input pin of the controller can receive a second set of inputs from the LED and sensor. The controller can then compare the first set of inputs against the second set of inputs to determine whether an acceptable monitoring condition exists.

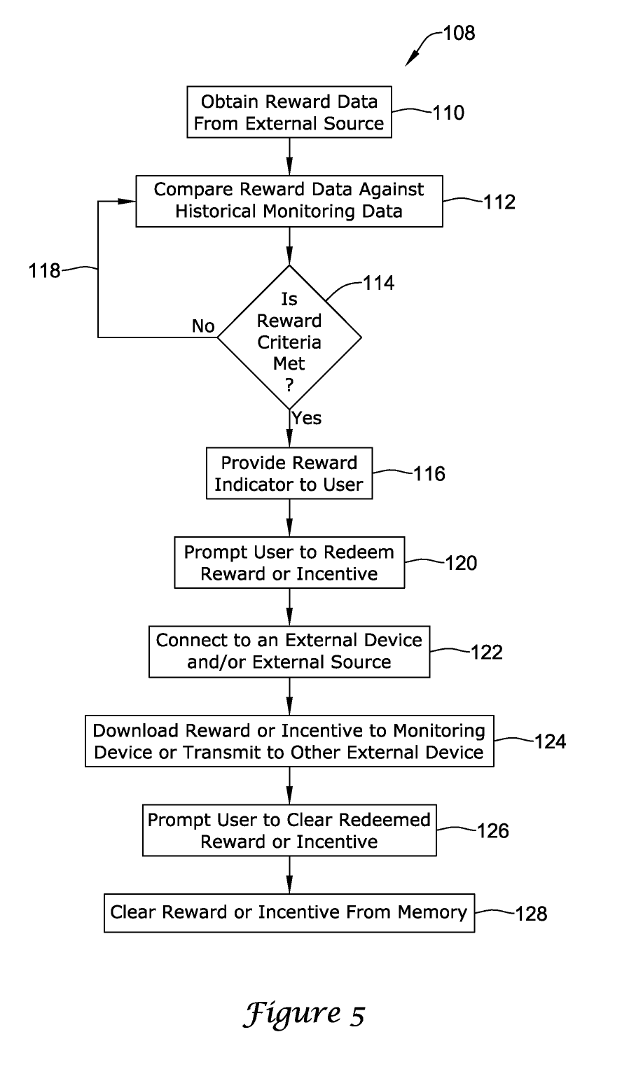

Referring now to FIG. 5, an illustrative method 108 of providing users with a reward or incentive for monitoring one or more medical parameters using a health monitoring device will now be described. The method may begin generally at block 110, with the step of obtaining reward data from an external source. Step 110 may include, for example, the process of downloading reward criteria data and/or one or more reward codes from a web-site or from another external device in communication with the monitoring device. In those embodiments in which the monitoring device is a blood glucose monitor, for example, the reward criteria may include one or more goals associated with the user's prescribed medical plan such as the lowering of blood pressure, increasing exercise levels, lowering blood sugar levels, etc. The reward data can further include one or more reward codes that can be used to reward the user for taking a number of glucose level readings within a certain period of time and/or for maintaining their glucose levels within a certain range. The reward criteria and reward codes will typically vary depending on the patient's particular medical condition, the type of monitoring device, the patient's gender and age, as well as other factors.

Once the monitoring device has obtained the reward criteria data at step 110, the monitoring device may next compare the reward criteria data against historical monitoring data previously obtained by the device, as indicated generally by block 112. In certain embodiments, for example, the comparison step 112 can include the step of comparing a glucose monitoring schedule containing the times and dates in which glucose levels are to be checked against one or more previously obtained glucose level readings stored within memory. Such step may be performed, for example, by the reward circuitry and/or from an external device in communication with the monitoring device. A date and time stamp may be provided in conjunction with the user's previously stored test data in order to compare the reward criteria with the actual measurements. The date and time stamp can be provided, for example, by the monitoring devices' internal clock or via an external source. In use, the internal or external clock can be used by the reward circuitry to determine the proper time to release the reward codes.

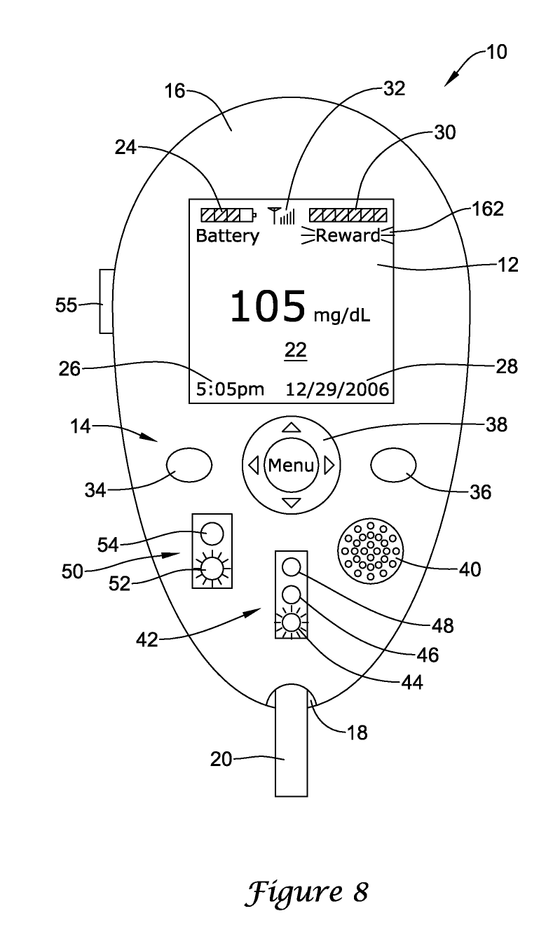

If at decision block 114 the reward circuitry determines that at least one goal of the reward criteria has been met, the monitoring device can be configured to provide the user with a reward indicator informing the user that a reward or incentive has been earned, as indicated generally by block 116. For example, if the user successfully maintains their glucose levels within a certain range for a predetermined period of time such as a week, the monitoring device can be configured to output a reward indicator 116 informing the user that they have achieved one of their goals. The reward indicator provided at step 116 may be in the form of a visual alert provided on the display screen of the monitoring device and/or an audible alert outputted by the speaker informing the user that they have earned a reward or incentive. In some embodiments, the visual alert may also be presented on the display screen of an external computing device. Tactile feedback (e.g. produced by an internal vibration element within the monitoring device) may also be provided notifying the user that a reward or incentive has been earned. An illustrative method of providing a visual alert to a user upon receiving a reward or incentive is described herein with respect to FIGS. 8-10.

If at decision block 114 the reward circuitry determines that the user has not successfully satisfied the reward criteria, the monitoring device may continue the step 112 of comparing the reward data against the user's historical data until the user satisfies one of the goals from the reward criteria, as indicated generally by arrow 118.

Once the user is notified of an earned reward, the monitoring device may further prompt the user to redeem the reward or incentive, as further indicated generally by block 120. For example, the monitoring device may output a message on the display screen informing the user that a reward has been earned along with instructions on how to redeem that reward. Once prompted, the user may then follow the instructions on the display screen, causing the monitoring device to connect to an external device, if necessary, in order to download a reward code, as indicated generally by block 122. For example, if at step 120 the monitoring device provides a URL address in which to redeem an earned reward, the user may then visit the web-site using either the monitoring device or some other external device in order to redeem the reward or incentive. As indicated generally by block 124, the user may then download the reward or incentive into the monitoring device and/or transmit the reward or incentive to another device. For example, the reward or incentive may be outputted to the display screen of a personal computer or hand-held computer as a pop-up advertisement. In some cases, the monitoring device may also track the time in which the reward was downloaded and/or redeemed.

Once the user has redeemed the reward or incentive, the monitoring device may then prompt the user to clear that reward or incentive from memory, as indicated generally by block 126. For example, and in some embodiments, the reward or incentive can be cleared manually by selecting one or more buttons on the user interface or by removing the batteries. In other embodiments, the internal clock within the monitoring device may be used to automatically determine when a reward or incentive is to be cleared. For example, the monitoring device may automatically delete the reward code after a certain period of time (e.g. one week) has elapsed, or when the reward code has been redeemed. The reward data can then erased from memory, as indicated generally by block 128.

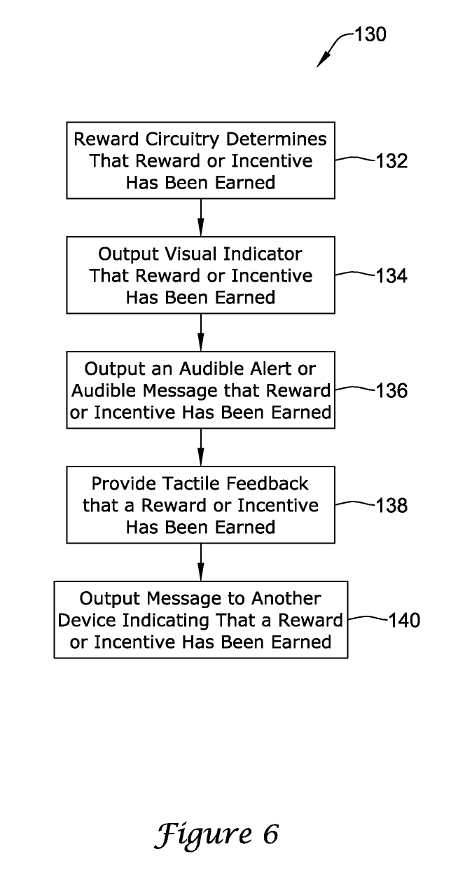

FIG. 6 is a flow diagram of an illustrative method 130 of providing a reward status indicator to a user using a health monitoring device. Method 130 may represent, for example, several illustrative steps to be performed in conjunction with block 116 described above with respect to FIG. 5. As shown in FIG. 6, the method 130 may begin generally at block 132 when the reward circuitry for the monitoring device determines that a reward or incentive has been earned by the user. Determination that a reward has been earned can be accomplished, for example, in a manner similar to that described above with respect to blocks 112 and 114 in FIG. 5, wherein the monitoring device compares the reward criteria data obtained from an external source and stored or from predetermined reward criteria stored within the monitoring device and then compares that data against historical monitoring data obtained by the device.

Once the monitoring device determines that one or more goals from the reward criteria have been met, the device can be configured to output a visual indicator notifying the user that a reward or incentive has been earned, as indicated generally by block 134. For example, at block 134 the monitoring device may provide a text message on the display screen of the monitoring device indicating that a reward or incentive has been earned. In some embodiments, for example, the monitoring device may alternate between displaying the user's most recent glucose level on the screen with a text message stating that a reward or incentive has or will be earned. In addition, one or more light sources may be illuminated on the monitoring device informing the user that the reward or incentive has or will be earned. For example, with respect to the illustrative monitoring device 10 described above with respect to FIG. 1, the LED's 52,54 may illuminate or change color in a particular manner notifying the user that a reward or incentive has been earned or is about to be earned.

As further indicated generally by block 136, the monitoring device may further output an audible alert or audible message informing the user that a reward or incentive has been earned. In certain embodiments, for example, the monitoring device may output an audible beep or tone from the speaker that can be distinguished from other alarm tones outputted by the device. A computer-simulated voice may further notify the user that a reward has been earned along with instructions on how to redeem that reward.

Other means for notifying the user that a reward or incentive has been earned may also be provided by the monitoring device. For example, and as indicated generally by block 138, the monitoring device can be configured to vibrate for a period of time, providing the user with tactile feedback that a reward or incentive has been earned. The monitoring device can also be configured to send an email message or instant message to another external device such as a computer or cellular telephone, as indicated generally by block 140. For example, the communications interface for the monitoring device can be used to send an email to user-specified email address and/or an SMS message to the user's cell phone, allowing the user to view, and in some cases redeem, the earned reward or incentive via the Internet or through the user's cell phone service provider.

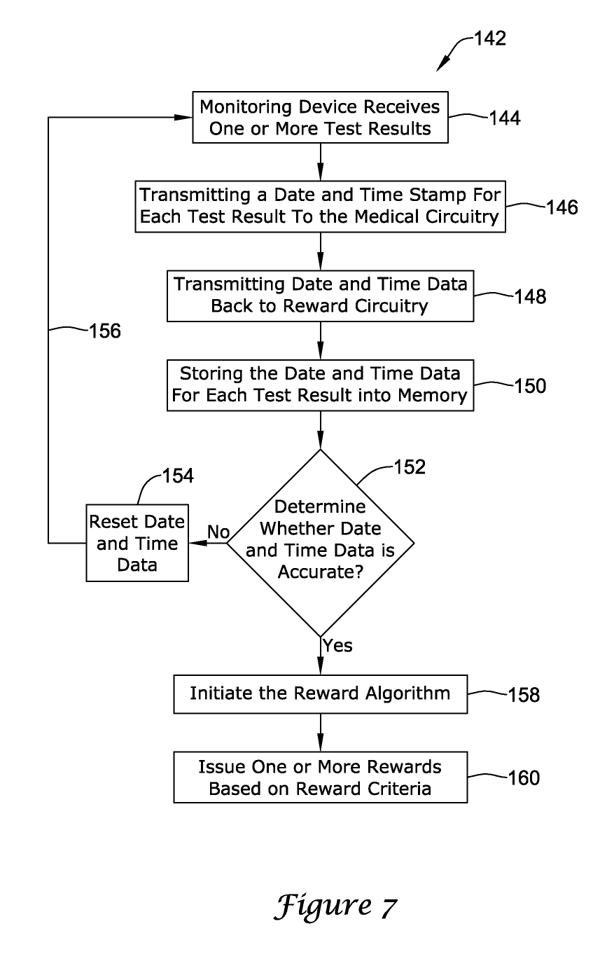

FIG. 7 is a flow diagram of an illustrative method 142 of triggering a reward or incentive using the internal clock circuitry of the health monitoring device. The method 142 may begin generally at block 144, in which the monitoring device receives on or more test results from the user. Block 144 may represent, for example, the step of obtaining one or more glucose level readings by inserting test strips into the monitoring device, or the step of taking one or more blood pressure readings using a blood pressure cuff. The test results received at block 144 will typically vary, however, depending on the type of monitoring device employed.

As each test reading is received and processed by the monitoring device, a date and time stamp corresponding to each test result can be transmitted to the medical circuitry and stored along with the medical data corresponding to each test reading, as indicated generally by block 146. The medical circuit may then send the date and time information for one or more of the test results back to the reward circuitry, as indicated generally by block 148. The reward circuitry may then store the date and time data for each test result into memory, as indicated generally by block 150.

At decision block 152, the reward circuitry may then determine whether the date and time data is accurate. Determination of the accuracy of the date and time data can be accomplished, for example, by comparing the date and time data of the actual test results with an external clock source used to synch the internal clock. If at block 152, the reward circuitry determines that the date and time stamps are not accurate, the reward circuitry may then reset the date and time data, as indicated generally by block 160. The process of receiving user input at block 144 can then be repeated, as indicated generally by arrow 156. If at decision block 152, however, the reward circuitry determines that the date and time data is accurate, the monitoring device can be configured to initiate the reward algorithm, as indicated generally by block 158. The monitoring device may then issue one or more rewards based on the reward criteria programmed within the reward criteria database, as indicated generally by block 160. The method 142 can then be repeated one or more times as each new test result is obtained.

FIGS. 8-10 are several perspective views of the monitoring device 10 of FIG. 1, showing several illustrative steps of providing a visual indicator to a user indicating that a reward or incentive has been earned. As can be seen in a first view in FIG. 8, the monitoring device 10 can be configured to blink a reward status indicator 162 on the display screen 22 when a reward or incentive has been earned, informing the user that a reward or incentive is available. Other means for proving the user with reward status information may also be provided as discussed herein, including the illumination of the LED's 52,54, the providing of an audible alert via the speaker 40 and/or the simultaneous display on a display screen of an external computing device.

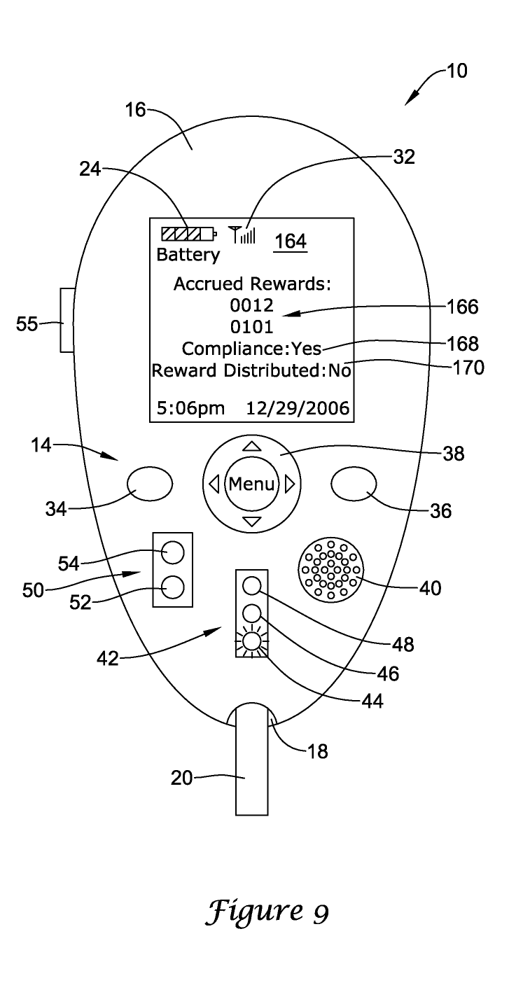

From the main screen depicted generally in FIG. 8, the user may then access a separate reward screen 164 providing the user with more information about the reward or incentive. For example, and as shown in a second view depicted in FIG. 9, the monitoring device may provide a list 166 of reward codes (e.g. “0012”, “0101”, etc.) for each reward earned, a message 168 indicating whether the user is currently in compliance with their prescribed medical plan, and a message 170 indicating whether the reward code or codes have been redeemed. Other information such as the current time and date may be further provided on the reward screen 164, if desired. Access to the reward screen 164 can be accomplished by selecting one or more buttons on the user interface 14 and/or remotely via an external computing device. Alternatively, and in other embodiments, the monitoring device 10 can be configured to automatically alternate between displaying the main display screen 22 and the reward screen 164, if desired.

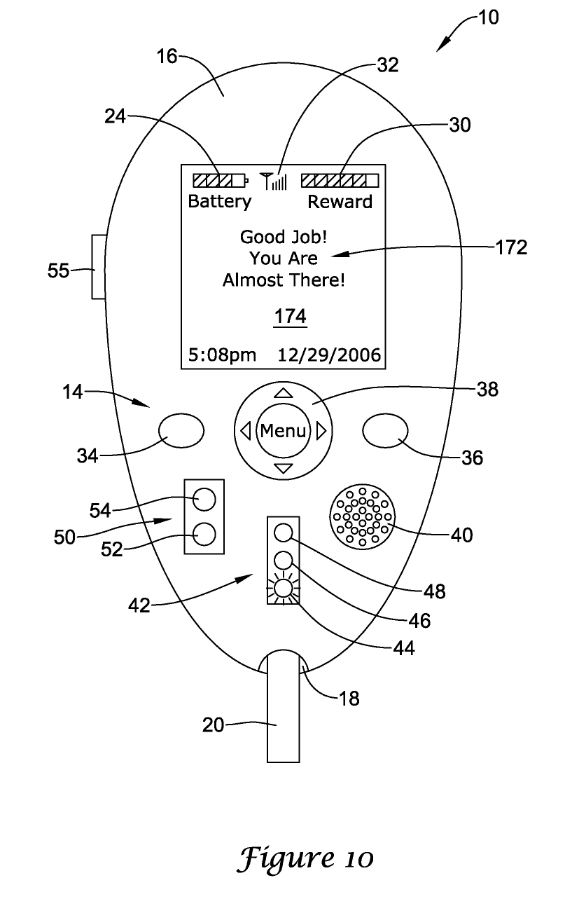

FIG. 10 is a perspective view showing a motivational message 172 displayed on a separate motivational display screen 174 of the monitoring device 10. As shown in FIG. 10, the monitoring device 10 can be configured to display one or more motivational messages 172 on the display screen 174 to encourage the user to comply with the goals of their medical plan. The motivational messages 172 can be downloaded into the monitoring device 10 from another external device, via a web-site on the Internet, or can be pre-programmed into the device 10.

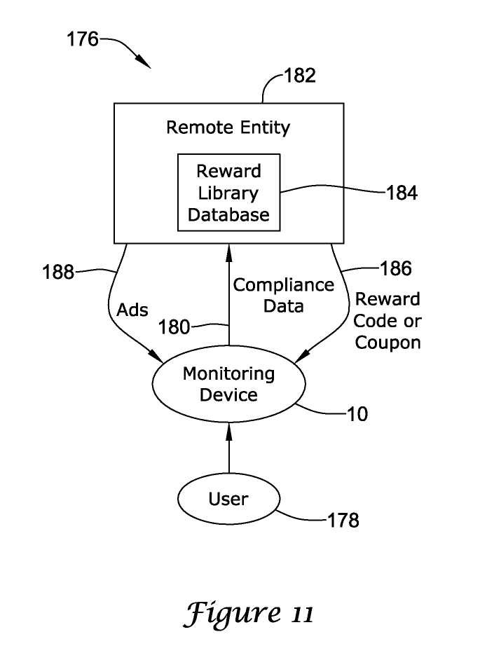

FIG. 11 is a diagrammatic view showing an illustrative reward system 176 for rewarding a user 178 of the health monitoring device 10 for monitoring one or more medical parameters. As shown in FIG. 11, the monitoring device 10 can be configured to transmit compliance data 180 such as the user's prior test readings to the computer system of one or more remote entities 182. An illustrative entity 182 may comprise, for example, a pharmacy, health-clinic, or other such business entity through which the user 178 normally purchases medical supplies such as insulin pumps, syringes, and test strips. Examples of other entities 182 can include, but are not limited to, insurance companies, self-insured employers, retailers, health-clubs, and fitness centers. The type of entity 182 will typically vary depending on the specific medical condition of the user 178 as well as the type of monitoring device 10 employed. For example, for individuals suffering from high blood pressure, the participating entity 182 may be a health club facility and the monitoring device 10 may be a blood pressure monitor.

The transmission of compliance data 180 to the participating entity 182 can occur via either a wireless or wired connection, and may vary depending on the type of remote entity 182 involved. For example, for a business such as a pharmacy or health-clinic, the transmission of compliance data 180 may occur automatically when the user enters the store or clinic, at regular intervals (e.g. once a day, once a week, once a month, etc.), when a reward or incentive has been earned, or some other desired criteria. The compliance data 180 can also be sent manually by the selection of a button or buttons on the monitoring device 10, or by some other deliberate action taken by the user. In some cases, the user may charge the entity 182 for the use of the compliance data 180.

In some embodiments, the compliance data 180 sent to the remote entity 182 may be converted to another format, if desired. For example, the compliance data 180 may be converted to a multimedia format such as MP3, MPEG, WAV, etc., and can be encrypted to prevent its interception from a third party.

The compliance data 180 transmitted to the remote entity 182 can be stored within a reward library database 184, which can then be used to generate one or more rewards or incentives based on the user' compliance with the entities' reward criteria, based on customer loyalty and/or patronage, as well as other factors. If, for example, the remote entity 182 is a medical supply store such as a pharmacy, the compliance data 180 transmitted can be used to generate rewards or incentives in the form of discount coupons for the purchase of medical supplies such as insulin pumps, syringes, test strips, etc. Other rewards or incentives such as cash-back rebates or credits may also be provided based on the user's compliance with the reward criteria established by the entity 182. For example, the reward or incentive may be in the form of discount coupons for diabetic test strips based on the user's glucose level readings. In some embodiments, rewards or incentives unrelated to the user's health may also be offered to the user 178. For example, the reward or incentive may be in the form of an access code that permits the user/customer to gain access to games, activities, or other such incentives on the remote entity's web-site.

The rewards or incentives generated by the remote entity 182 can be transmitted back to the user's monitoring device 10 and redeemed in a manner similar to that discussed above with respect to FIG. 5. As indicated generally by arrow 186, for example, the participating entity 182 may transmit a reward code or coupon to the user's monitoring device 10, which can then be redeemed at a later time at the remote entity 182, at another store, on a web-site, and/or at some other location. Rewards or incentives generated by the participating entity 182 may also be transmitted to another location for redemption. For example, the reward or incentive may be transmitted to a cash register within the entity's store that can be redeemed automatically at the point of sale, or to a web-site that can be later accessed by the user to obtain an on-line discount or other such incentive. In use, reward codes provided to the monitoring device 10 can be used as part of the remote entity's business plan to achieve customer retention and increase customer loyalty.

The remote entity 182 may further transmit one or more advertisements to the user's monitoring device 10, as indicated generally by arrow 188, informing the user 178 of any specials, price reductions, bulk discounts, and/or new products that may be available for purchase. The advertisements 188 can be targeted based on the user's specific medical condition, or can be provided to a larger subset of the population. For example, targeted advertisements can be provided to diabetics informing them of certain diabetic products that are on sale and/or any new products that may be of interest. For individuals suffering from asthma, the advertisements may correspond to other related products such as allergy relief medicines and/or air filtration products. In some cases, the advertisements 188 provided to the user may be based on the user's compliance data 180.

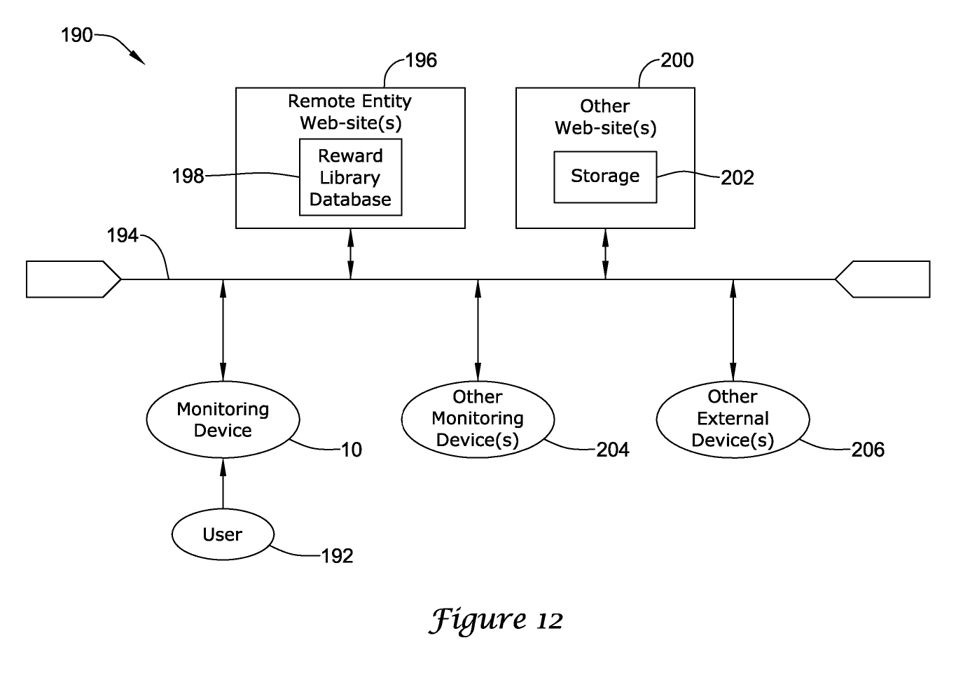

FIG. 12 is a diagrammatic view showing an illustrative on-line reward system 190 for rewarding a user 192 of the health monitoring device 10 for monitoring one or more medical parameters. As shown in FIG. 12, the monitoring device 10 can be connected to one or more on-line components via an Internet or intranet connection 194, including one or more remote entity websites 196 each having a reward library database 198 that can be accessed by the user 192 to obtain various product and store information, advertisements, discount coupons, as well as other information. An example remote website 196 may comprise, for example, an on-line pharmacy or medical supply store having a specialized rewards web-page that can be accessed by the user to view and/or redeem one or more earned rewards or incentives. The monitoring device 10 may be further linked to one or more other web-sites 200 each including storage 202 for storing information about the user's compliance with their prescribed medical plan, any past rewards or incentives that have been received, the anticipated time when another reward or incentive will be received, customer identifying information, as well as other information. In certain embodiments, for example, one of the other web-sites 200 may comprise the user's own personal web-site or the web-site of their healthcare provider or health insurance provider.

As can be further seen in FIG. 12, one or more other monitoring devices 204 and/or other external devices 206 can also be linked to the monitoring device 10 as well as the remote entity websites 196 and other web-sites 200 via the Internet or an intranet connection 194. Other monitoring devices 204 that can be connected can include, but are not limited to, blood pressure meters, blood oxygen monitors, insulin pumps, continuous glucose monitors, prosthetic devices, shoes, scales, pedometers, exercise equipment, heart monitors, and spirometers. Other external devices 206 that can be connected can include, but are not limited to, personal computers, laptop computers, hand-held computers, cellular telephones, pagers, television set or cable boxes, video game consoles, and digital media players. In one illustrative embodiment, for example, a television or cable set box can be connected to the monitoring device 10 via the Internet 194, allowing the user 192 to transmit and receive data and messages back and forth between the monitoring device 10 and one or more of the other components 196,200,204,206.

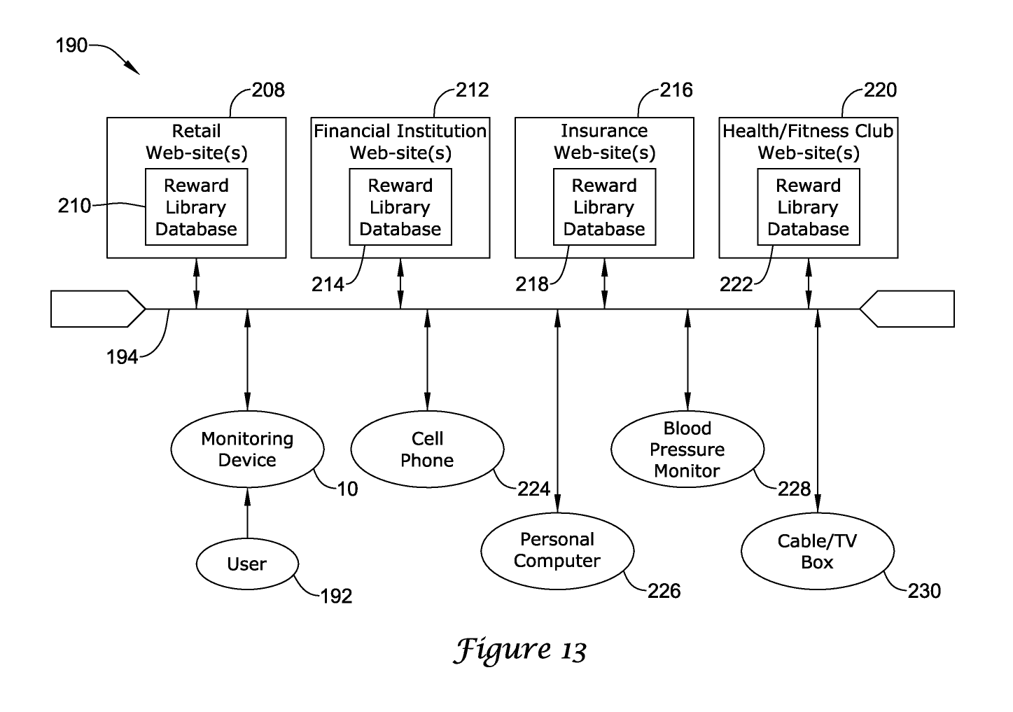

FIG. 13 is a diagrammatic view showing an illustrative implementation of the on-line reward system 190 of FIG. 12. As shown in FIG. 13, the monitoring device 10 can be connected to one or more retail web-sites 208 each including a reward library database 210 that can be accessed by the user 192 to obtain various product and store information, advertisements, discount coupons, as well as other information. The monitoring device 10 can also be connected to one or more financial institution web-sites 212 each including a reward library database 214. An example financial institution web-site may include a credit card company or bank in which the user 192 conducts business with. Based on the user' compliance with the reward criteria within the reward library database 214, the credit card company or bank may provide the user with a reward or incentive such as a credit or rebate to their account.

The monitoring device 10 can also be connected to one or more insurance web-sites 216 each including a reward library database 218. In certain embodiments, for example, insurance web-sites 216 may include the web-site of the use's heath insurance provider. Based on the user's compliance with the reward criteria within the reward library database 218, the insurance provider may then provide the user with a reward or incentive such as a reduction in health insurance premiums or co-pays. In similar fashion, the monitoring device 10 can be connected to one or more health/fitness club web-sites each including a reward library database 222. Based on the user's compliance with the reward criteria within the reward library database 222, the health/fitness club may provide the user with a reward or incentive such as the reduction in membership fees. As further shown in FIG. 13, the monitoring device 10 can be connected to one or more other devices including, but not limited to, a cellular telephone 224, personal computer 226, blood pressure monitor 228 and/or a cable/television set box 230.

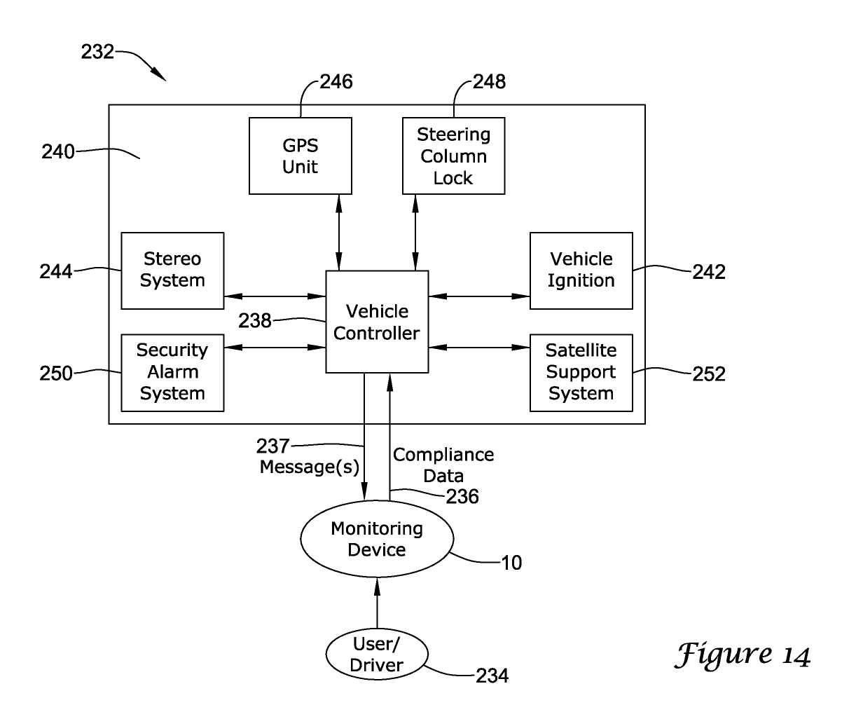

FIG. 14 is a diagrammatic view showing an illustrative vehicle reward system 232 for rewarding a driver 234 that uses the monitoring device 10 for monitoring one or more medical parameters. As shown in FIG. 14, the monitoring device 10 can be configured to transmit compliance data 236 to a vehicle controller 238 of a vehicle 240, which can be used by the controller 238 to decide whether to permit the driver 234 to start the vehicle 240 based on the driver's current medical condition and/or the time or times of their most recent readings. For diabetic patients, for example, the monitoring device 10 can be configured to transmit the last few glucose level readings to the vehicle controller 238, which can then be used by the controller 238 to determine whether to activate the vehicle's ignition system 242 and/or steering column lock 248 based on the compliance data 236 received from the monitoring device 10. If, for example, the drivers' glucose level is determined to be too high (e.g. above 101), the vehicle controller 238 can be configured to deactivate the vehicle ignition system 242 and/or steering column lock 248, preventing the driver 234 from operating the vehicle 240 until their levels are brought within a certain range. In such event, the vehicle controller 238 may transmit a message 237 back to the monitoring device 10 and/or some other display screen such as one found on a navigational system notifying the driver 234 that their current glucose levels are too high to permit the safe operation of the vehicle 240. The monitoring device 10 can then prompt the driver 234 to take another measurement prior to operating the vehicle 240.

The compliance data 236 received by the vehicle controller 238 can also be outputted to other components of the vehicle 240. For example, and as further shown in FIG. 14, the vehicle controller 238 can be connected to the vehicle's audio system 244, a GPS navigational unit 246, the vehicle's steering column lock 248, the vehicle's security alarm system 250 and/or a satellite support system 252. The vehicle's audio system 244, for example, can be utilized to generate an audible alarm notifying the driver 234 to take a measurement, providing the user with a warning if their levels are out of range, etc. The vehicle's security alarm system 250, in turn, can be used to provide other individuals with an indication that the driver 234 may require assistance. For example, the vehicle security alarm system 250 may cause the emergency lights on the vehicle to automatically flash if the driver's glucose levels fall within a certain range. In those vehicles equipped with a satellite support system 252, such information could also be relayed to an operator in order to provide the operator with more detailed information on what emergency services to dispatch.

In those vehicles equipped with a GPS unit 246, the monitoring device 10 may be further used in conjunction with the vehicle's GPS mapping software to provide the driver 234 with directions to the closest medical supply store for purchasing needed supplies, the location of the closest emergency facility, etc. In some embodiments, the GPS mapping software may also be used to notify the driver 234 where to redeem a reward or incentive that has or will be earned.

The monitoring device 10 may use its internal clock to determine the last time a measurement was taken, which can then be transmitted to the vehicle 240 as a part of the compliance data 236 and compared against the vehicle's own internal clock. Comparison of the monitoring device internal clock with the vehicle clock can be accomplished in a manner similar to that described above with respect to FIG. 7, by comparing the user input against reward criteria within the reward circuitry to determine the accuracy of the time measurements. The monitoring device 10 can be programmed to permit the driver 234 to operate the vehicle only if a reading has been obtained within a certain period of time. For example, the monitoring device can be programmed to only permit the driver 234 to operate the vehicle 240 if a glucose level reading has been obtained within the past four hours, within the past 2 hours, or other such time interval.

Having thus described several embodiments of the present invention, those of skill in the art will readily appreciate that other embodiments may be made and used which fall within the scope of the claims attached hereto. It will be understood that this disclosure is, in many respects, only illustrative. Changes can be made with respect to various elements described herein without exceeding the scope of the invention.

What is claimed is:

1. A method of providing individuals with a reward or incentive for monitoring one or more medical parameters, the method comprising the steps of:
providing a health monitoring device adapted to sense one or more medical parameters relating to a user's health, the health monitoring device including reward circuitry adapted to run an algorithm or routine for encouraging the monitoring of one or more medical parameters;
receiving reward criteria data into the health monitoring device;
comparing the reward criteria data against one or more medical parameters obtained by the health monitoring device;
determining whether one or more goals of the reward criteria data have been satisfied;
providing a reward indicator notifying the user when at least one of said one or more goals have been achieved;
prompting the user to redeem one or more rewards or incentives based at least in part on their compliance with said one or more goals; and
downloading the reward or incentive into a storage memory of the health monitoring device and/or transmitting the reward or incentive to one or more other devices.

2. The method of claim 1, wherein said step of receiving reward criteria data includes the step of downloading the reward criteria from a web-site and/or other remote device in communication with the health monitoring device.

3. The method of claim 1, wherein said step of comparing the reward criteria data against one or more medical parameters includes the step of using an internal clock of the monitoring device to determine a date and/or time associated with each obtained medical parameter.

4. The method of claim 1, wherein said step of determining whether one or more goals of the reward criteria data have been satisfied includes the step of comparing a date and time stamp associated with each medical parameter with time and date data from an external clock source.

5. The method of claim 1, wherein said step of providing a reward status indicator includes the step of outputting a visual alert on a display screen.

6. The method of claim 5, wherein said health monitoring device is adapted to alternate between displaying the visual alert and one or more medical parameters on the same display screen.

7. The method of claim 1, wherein said step of providing a reward status indicator includes the step of illuminating one or more lights on the health monitoring device.

8. The method of claim 7, wherein said step of illuminating one or more lights on the health monitoring device includes:
illuminating a first array of lights on the health monitoring device adapted to provide feedback on the user's compliance with one or more goals of the reward criteria; and
illuminating a second array of lights on the health monitoring device adapted to provide feedback on any rewards or incentives that have or will be earned based at least in part on based on the user's compliance with said one or more goals.

9. The method of claim 8, wherein said step of illuminating the first array of lights on the health monitoring device includes the steps of:
illuminating a first light indicating when the user is in full compliance with said one or more goals; and
illuminating one or more other lights indicating when the user is not in compliance with said one or more goals.

10. The method of claim 7, wherein said step of illuminating one or more lights on the health monitoring device includes the step of altering the color and/or blink rate of at least one of said lights.

11. The method of claim 1, wherein said step of providing a reward status indicator includes the step of outputting an audible alarm through a speaker.

12. The method of claim 11, further including the step of adjusting the pitch and/or loudness of the audible alarm based at least in part on the user's compliance with said one or more goals.

13. The method of claim 1, wherein said step of providing a reward status indicator includes the step of providing the user with tactile feedback with a vibration element.

14. The method of claim 1, wherein said step of prompting the user to redeem one or more rewards or incentives includes the step of outputting a message on a display screen informing the user that a reward or incentive has been earned along with instructions on how to redeem the reward or incentive.

15. The method of claim 1, further comprising the step of prompting the user to clear the reward or incentive from memory.

16. The method of claim 1, further comprising the step of automatically erasing the reward or incentive from memory.

17. The method of claim 1, further comprising the step of outputting one or more motivational and/or advertising messages on a display screen based at least in part on the user's compliance with said one or more goals.

18. A system for encouraging the monitoring of one or more medical parameters by a user, the system comprising:
a health monitoring device adapted to sense one or more medical parameters relating to a user's health, the health monitoring device including reward circuitry adapted to run an algorithm or routine for encouraging the monitoring of said one or more medical parameters; and
at least one remote device in communication with the health monitoring device.

19. The system of claim 18, wherein said at least one remote device includes the computer system of a remote entity.

20. The system of claim 19, wherein said remote entity is a pharmacy or medical supply store.

21. The system of claim 18, wherein said at least one remote device is a vehicle controller.

22. The system of claim 18, wherein said at least one remote device includes a web-site.

23. The system of claim 18, wherein said at least one remote device includes one or more monitoring devices.

24. The system of claim 18, wherein said at least one remote device is in communication with the health monitoring device via a wired or wireless connection.

25. The system of claim 18, wherein said at least one remote device is in communication with the health monitoring device via an Internet or intranet connection.

26. The system of claim 18, wherein said at least one remote device includes a reward database containing one or more rewards or incentives.

27. The system of claim 26, wherein said reward database includes one or more programmed reward criteria.

28. The system of claim 18, wherein the health monitoring device is adapted to transmit compliance data to the at least one remote device, the compliance data including one or more readings obtained from the health monitoring device.

29. The system of claim 28, wherein said at least one remote device is adapted to transmit one or more reward codes to the health monitoring device based on said compliance data.

30. A health monitoring device, comprising:
medical circuitry adapted to sense one or more medical parameters;
reward circuitry including a reward algorithm or routine for encouraging the monitoring of said one or more medical parameters;
a communications interface for transmitting and/or receiving reward data and/or medical data back and forth between the health monitoring device and one or more remote devices in communication with the health monitoring device;
at least one display panel including a display screen for displaying reward data; and
a user interface.

31. The health monitoring device of claim 30, further including a storage memory adapted to store one or more sensed medical parameters along with a date and time stamp corresponding to each sensed medical parameter.

32. The health monitoring device of claim 30, wherein the reward circuitry includes a reward code database containing one or more programmed reward codes.

33. The health monitoring device of claim 30, wherein the reward circuitry includes a reward criteria database containing one or more programmed goals.

34. The health monitoring device of claim 33, wherein the reward algorithm or routine is adapted to formulate a reward or incentive based at least in part on the user's compliance with said one or more programmed goals.

35. The health monitoring device of claim 33, wherein the health monitoring device further includes:
a first array of light sources adapted to provide feedback on the user's compliance with said one or more programmed goals; and
a second array of light sources adapted to provide the user with feedback on any rewards or incentives that have or will be earned based on the user's compliance with said one or more programmed goals.

36. The health monitoring device of claim 35, further comprising a means for determining the operational status of each light source within the first and/or second array of light sources.

37. The health monitoring device of claim 30, wherein the reward circuitry is optically isolated from the medical circuitry.

38. The health monitoring device of claim 30, wherein the health monitoring device further includes a motivation and/or advertising message database containing one or more motivational and/or advertising messages.

39. The health monitoring device of claim 38, further comprising an interface for programming the motivational and/or advertising messages within said motivation and/or advertising message database.

40. The health monitoring device of claim 30, wherein the health monitoring device is a blood glucose meter.
